(19) United States
(12) Patent Application Publication
(10) Pub. No.: US 2016/0340815 A1
(43) Pub. Date: Nov. 24, 2016

(54) LIQUID-ABSORBENT COMPOSITE NONWOVEN FABRIC AND ARTICLES THEREOF

(71) Applicant: 3M INNOVATIVE PROPERTIES COMPANY, Saint Paul, MN (US)

(72) Inventors: CARY A. KIPKE, AUSTIN, TX (US); JOHN J. ROGERS, ST. PAUL, MN (US); DANIEL J. ZILLIG, WOODBURY, MN (US); RANDY L. CHRISTIANSEN, POMPANO, FL (US); DANIEL E. JOHNSON, MARINE ON ST. CROIX, MN (US)

(21) Appl. No.: 15/106,864

(22) PCT Filed: Dec. 17, 2014
(86) PCT No.: PCT/US2014/070750

§ 371 Date: Jun. 21, 2016

Related U.S. Application Data
(60) Provisional application No. 61/921,166, filed on Dec. 27, 2013.

(51) Int. Cl. D04H 1/4382; D04H 1/56; D04H 1/4358; A61F 13/00; A61F 13/53

(52) U.S. Cl. CPC D04H 1/4382; A61F 13/00042; A61F 13/00008; A61F 13/00029; A61F 13/53; D04H 1/4358; D04H 1/56; D10B 2509/022; D10B 2509/026

(57) ABSTRACT

A composite nonwoven fabric and articles comprising the composite nonwoven fabric are provided. The composite nonwoven fabric comprises a population of meltblown fibers comprising an aliphatic polyether thermoplastic polyurethane polymer having at least about 80% (by weight) polyalkylene oxide and a population of staple fibers intermixed and entangled therewith.

### CROSS REFERENCE TO RELATED APPLICATIONS

This claims priority to U.S. Provisional Patent Application No. 61/921,166, filed Dec. 27, 2013, the disclosure of which is incorporated by reference in its entirety herein.

### BACKGROUND

Porous webs are in widespread use in applications such as filtration of particulates and removal of oil from water, absorption of fluid discharges from a human body, and as acoustic or thermal insulation. Some porous webs have been made from thermoplastic resins using melt-blowing techniques of the type described in Report No. 4364 of the Naval Research Laboratories, published May 25, 1954, entitled “Manufacture of Super Fine Organic Fibers” by Van A. Wente et al., which is incorporated herein by reference in its entirety.

In addition, composite webs may be formed using a mixture of meltblown fiber webs and other polymeric fibers (e.g., staple fibers), as described in U.S. Pat. No. 6,827,764, granted to Springett et al.; U.S. Pat. No. 4,118,531, granted to Hauser; and U.S. Pat. No. 4,908,263, granted to Reed et al.; and U.S. Patent Application Publication No. 2008/0318024; which are all incorporated herein by reference in their entirety.

Bodily fluids typically have a variety of solutes (e.g., proteins, carbohydrates, salts) dissolved therein. In addition, lavage solutions (e.g., saline, buffered saline, Ringer's solution) that are used to moisten and/or rinse wound sites typically contain solutes (e.g., sodium chloride, sodium lactate) dissolved therein. There is a need for materials and articles to absorb aqueous liquids such as, for example, bodily fluids and/or aqueous solutions that are used to treat wound sites.

### SUMMARY

The present disclosure generally relates to compositions and articles for absorbing an aqueous liquid. In particular, the present disclosure relates to a composite meltblown nonwoven fabric comprising a population of meltblown fibers that are intermixed and entangled with a population of staple fibers. The composite fabric is soft, compliant, has excellent moisture-absorbent properties and maintains its structural integrity when hydrated with an aqueous liquid.

The composite nonwoven fabric of the present disclosure can comprise a population of meltblown fibers that are capable of absorbing an amount of aqueous liquid equal to at least about 1 times its weight, the meltblown fibers being intermixed and entangled with staple fibers. In any embodiment, the composite nonwoven fabric is capable of absorbing an amount of aqueous liquid equal to at least about 1 times its weight to at least about 6 times its weight. In any embodiment, the meltblown fibers can comprise a thermoplastic polyurethane polymer. In contrast to typical elastomeric polyurethanes that are used in meltblown processes, the thermoplastic polyurethane fibers of the present disclosure are highly water-absorbent (e.g., the thermoplastic polyurethanes of the present disclosure are able to absorb an amount of water up to several times their weight.

In one aspect, the present disclosure provides a composite nonwoven fabric. The composite nonwoven fabric can comprise a population of meltblown fibers and a population of staple fibers intermixed and entangled therewith. The meltblown fibers comprise an aliphatic polyether thermoplastic polyurethane polymer having at least about 80% (w/w) polyalkylene oxide. In any embodiment, the meltblown fibers comprise an aliphatic polyether thermoplastic polyurethane polymer having at least about 90% (w/w) polyalkylene oxide.

In another aspect, the present disclosure provides an article. The article can comprise a composite nonwoven fabric comprising a population of meltblown fibers and a population of staple fibers intermixed and entangled therewith. The meltblown fibers comprise an aliphatic polyether thermoplastic polyurethane polymer having at least about 80% (w/w) polyalkylene oxide. In any embodiment, the meltblown fibers comprise an aliphatic polyether thermoplastic polyurethane polymer having at least about 90% (w/w) polyalkylene oxide.

In any of the above embodiments, the staple fibers can be selected from the group consisting of cellulose fibers, regenerated cellulose fibers, polyester fibers, polypeptide fibers, hemp fibers, flax fibers, nylon fibers, and a mixture of any two or more of the foregoing fibers.

In any of the above embodiments, at least a portion of the population of staple fibers is thermally bonded to the meltblown fibers.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

As used herein, “a,” “an,” “the,” “at least one,” and “one or more” are used interchangeably. Thus, for example, “a” fiber can be interpreted to mean “one or more” fibers.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

Additional details of these and other embodiments are set forth in the accompanying drawings and the description below. Other features, objects and advantages will become apparent from the description and drawings, and from the claims.

### BRIEF DESCRIPTION OF DRAWINGS

FIG. 1 is a schematic view of one embodiment of a process for making a web comprising meltblown fibers and staple fibers according to the present disclosure.

FIG. 2 is a perspective view of one embodiment of an article comprising a composite meltblown nonwoven fabric according to the present disclosure.

FIG. 3 is a perspective view of one embodiment of an article comprising a plurality of composite meltblown nonwoven fabric layers according to the present disclosure.

FIG. 4 is a perspective view of one embodiment of an article comprising a composite meltblown nonwoven fabric layer bonded to a backing layer according to the present disclosure.

FIG. 5 is a perspective view of one embodiment of an article comprising a plurality of composite meltblown nonwoven fabric layers bonded to a backing layer according to the present disclosure.

While the above-identified drawing figures set forth several embodiments of the disclosure, other embodiments are also contemplated, as noted in the discussion. In all cases, this disclosure presents the invention by way of representation and not limitation. It should be understood that numerous other modifications and embodiments can be devised by those skilled in the art, which fall within the scope and spirit of the principles of the invention. The figures may not be drawn to scale.

### DETAILED DESCRIPTION

Before any embodiments of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “connected” and “coupled” and variations thereof are used broadly and encompass both direct and indirect connections and couplings. Further, “connected” and “coupled” are not restricted to physical or mechanical connections or couplings. It is to be understood that other embodiments may be utilized and structural or logical changes may be made without departing from the scope of the present disclosure. Furthermore, terms such as “front,” “rear,” “top,” “bottom,” and the like are only used to describe elements as they relate to one another, but are in no way meant to recite specific orientations of the apparatus, to indicate or imply necessary or required orientations of the apparatus, or to specify how the invention described herein will be used, mounted, displayed, or positioned in use.

“Meltblown”, as used herein, refers to a process of extruding a molten material through a plurality of orifices to form filaments while contacting the filaments with air or other attenuating fluid to attenuate the filaments into fibers, and thereafter collecting a layer of the attenuated fibers.

“Meltblown fibers” means fibers prepared by the meltblown process.

“Diameter” when used with respect to a fiber means the fiber diameter for a fiber having a circular cross section, or, in the case of a non circular fiber, the length of the longest cross-sectional chord that may be constructed across the width of the fiber.

“Effective Fiber Diameter” when used with respect to a collection of fibers means the value determined according to the method set forth in Davies, C. N., “The Separation of Airborne Dust and Particles”, Institution of Mechanical Engineers, London, Proceedings 1B, 1952 for a web of fibers of any cross-sectional shape be it circular or non-circular.

“Self-supporting”, as used herein, refers to a web having sufficient strength so as to be handleable by itself using reel-to-reel manufacturing equipment without substantial tearing or rupture.

“Staple fibers”, as used herein, refers to fibers that have determinate length, generally between 5-200 mm. These fibers may have a crimp imparted to them.

The present disclosure relates generally to liquid-absorbent fabrics and articles comprising the liquid-absorbing fabrics. In particular, the present disclosure relates to compositions and articles that absorb aqueous liquids. The inventive compositions disclosed herein are highly water-absorbent and the absorbency is not substantially diminished by a presence of solutes in the aqueous liquid. Thus, the inventive articles comprising the compositions are particularly useful for absorbing aqueous biological fluids.

Highly-absorbent, biocompatible materials are desirable for use in liquid management. They can be particularly useful in managing biological liquids (e.g., serum, blood, wound exudate, amniotic fluid, sweat, urine). The use of highly-absorbent, biocompatible materials in wound dressings may preserve a moist environment that facilitates wound healing, while also removing excess fluid that otherwise might lead to tissue maceration. Preferably, the absorbency of the highly-absorbent material is not substantially decreased by a presence of dissolved solutes (e.g., salts) in the liquid to be managed.

Highly-absorbent polymeric materials can be used to absorb water. For example, LUBRIZOL Life Science Polymers Wickliffe, Ohio) provides polyether-based hydrogel thermoplastic polyurethane (TPU) polymeric resins that can be used to absorb or transport moisture. However, TPU's comprising a relatively-high (e.g., at least about 80%) weight percentage of polyalkylene oxide; which can be solution cast, coated, or extruded; are known to form weak gels when hydrated, resulting in materials that may have less physical integrity than desired under certain circumstances. In addition, laminates comprising thin films of such highly-absorbent TPU's are susceptible to delamination when hydrated because of the significant swelling that occurs upon hydration. It is now known that such TPU's can be extruded in a meltblown process that blends the meltblown fibers with staple fibers to form a nonwoven fabric having highly-desirable liquid absorbent properties as well as improved structural integrity when hydrated. In addition, the absorbency of the resulting composite nonwoven fabric is not substantially decreased by a presence of dissolved solutes in an aqueous liquid.

In one aspect, the present disclosure provides a composite nonwoven fabric. The fabric comprises a population of meltblown fibers comprising an aliphatic polyether thermoplastic polyurethane (TPU) polymer having at least about 80% (w/w) polyalkylene oxide and a population of staple fibers intermixed and entangled therewith. In any embodiment, the fabric comprises a population of meltblown fibers comprising an aliphatic polyether thermoplastic polyurethane polymer having at least about 85% (w/w) polyalkylene oxide and a population of staple fibers intermixed and entangled therewith. In any embodiment, the fabric comprises a population of meltblown fibers comprising an aliphatic polyether thermoplastic polyurethane (TPU) polymer having at least about 90% (w/w) polyalkylene oxide and a population of staple fibers intermixed and entangled therewith.

In addition to being intermixed and entangled with the meltblown fibers, in any embodiment, at least a portion (e.g., at least 10%, at least 20%, at least 30%, at least 40%, at least 50%, at least 60%, at least 70%, at least 80%, at least 90%) of the staple fibers can be thermally bonded to the meltblown fibers. Without being bound by theory, it is thought this thermal bonding may be facilitated by the use of a thermoplastic polyurethane polymer (e.g., TECOPHILIC hydrogel thermoplastic urethane TG-2000 sold by The Lubrizol Corporation; Wickliffe, Ohio) that solidifies relatively slowly compared to other thermoplastic polyurethanes used to make meltblown fibers

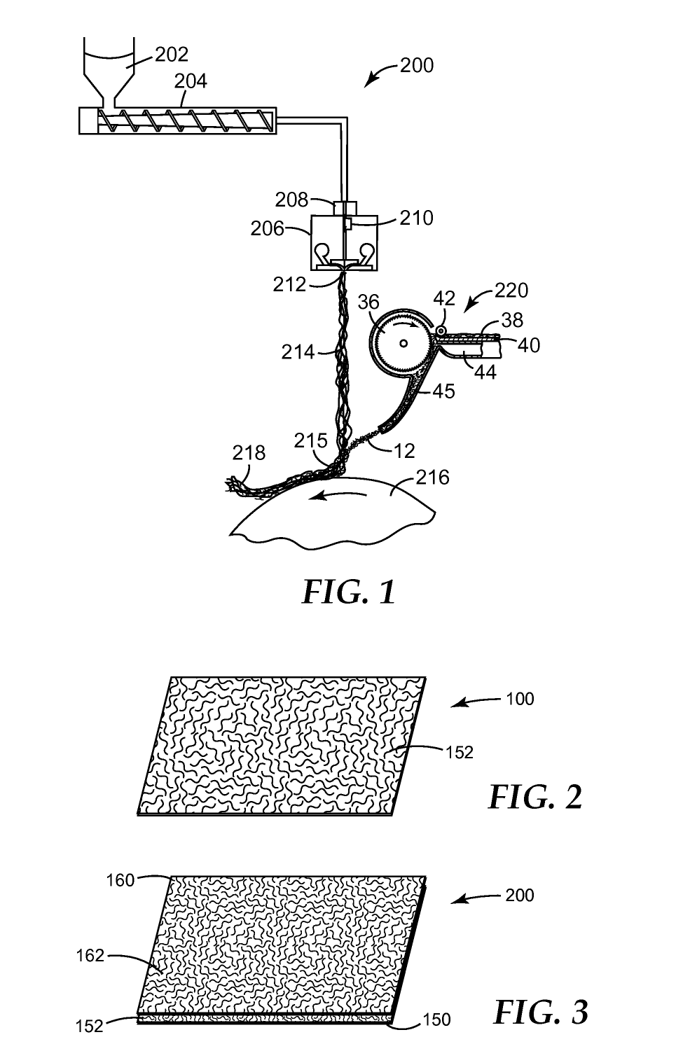

The composite nonwoven fabric of the present disclosure can be produced using the meltblown process described in U.S. Pat. No. 4,118,531. FIG. 1 shows one embodiment of an apparatus 200 for making an article according to the present disclosure. Molten fiber-forming polymeric material fed from hopper 202 and extruder 204 enters meltblowing die 206 via inlet 208, flows through die cavity 210, and exits die cavity 210 of orifices arranged in line across the forward end of die cavity 210 and in fluid communication with die cavity 210 (in one embodiment, die cavity 210 is in fluid communication with the orifices by means of a conduit or conduits, not shown in FIG. 1). The molten fiber-forming material is thus extruded from the orifices so as to form filaments 212. A set of openings is provided through which a gas, typically heated air, is forced at very high velocity, so as attenuate the filaments 212 into fibers, which form air-borne stream 214 of melt blown meltblown fibers. In a particular embodiment, the above-described apparatus comprises a single extruder, a single die, and a single die cavity.

Staple fibers 12 may be introduced into the stream 214 of meltblown fibers through the use of exemplary apparatus 220 shown in FIG. 1. Such an apparatus provides a lickerin roll 36 which is disposed near the melt-blowing apparatus. A collection 38 of staple fibers (typically a loose, nonwoven web such as prepared on a garnet machine or “Rando-Webber”), is propelled along a table 40 under a drive roll 42 where the leading edge engages against the lickerin roll 36. The lickerin roll 36 turns in the direction of the arrow and picks off fibers from the leading edge of the collection 38, separating the fibers from one another. The picked fibers are conveyed in an air stream through an included trough or duct 45 and into the stream 214 of melt blown meltblown fibers where they become mixed with the meltblown fibers. The air stream may be generated inherently by rotation of the lickerin roll, or the air stream may be augmented by use of an auxiliary fan or blower operating through a duct 44.

The mixed intermingled stream 215 of staple fibers and meltblown fibers then continues to collector 216 where the mixed fibers form a self-supporting web (i.e., nonwoven fabric). The collector 216 typically is a finely perforated screen, which may comprise a closed-loop belt, a flat screen or a drum or cylinder. A gas-withdrawal apparatus may be positioned behind the screen to assist in depositing the fibers and removing the gas. The resulting web 218 may be peeled off the collector and wound into a storage roll and may be subsequently processed in cutting, handling, or molding operations.

The inventors have discovered that using a meltbown process to cast fibers comprising an aliphatic polyether thermoplastic polyurethane polymer having about 90% (w/w) polyalkylene oxide (PAO) can result in the merging of the individual fibers to form a continuous or semi-continuous film, rather than a porous fabric. The inventors further have discovered that the introduction of staple fibers into a stream of the meltblown PAO-containing polymer surprisingly reduces or prevents the film formation by the PAO-containing polymer in the resulting nonwoven fabric. In addition, the staple fibers provide strength and support for the fabric when the meltblown fibers are hydrated with an aqueous liquid.

Aliphatic polyether TPU polymers are known in the art. Aliphatic polyether TPU polymers that are suitable to make the nonwoven fabrics of the present disclosure include polymers that comprise block subunits of polyalkylene oxides. Suitable polyalkylene oxides include, for example, polyethylene oxide (PEO), polypropylene oxide (PPO) or mixtures thereof. In any embodiment, the polymer resin used to form the nonwoven fabric is a medical grade TPU polymer. A nonlimiting example of a medical grade TPU polymer suitable to form nonwoven fabrics of the present disclosure is the TECOPHILIC hydrogel TPU (Part number TG-2000) sold by The Lubrizol Corporation (Wickliffe, Ohio). In any embodiment, the block subunits of polyalkylene oxide in the TPU polymer can have a formula weight of about 6,000 daltons to about 20,000 daltons. In any embodiment, the block subunits of polyalkylene oxide in the TPU polymer can have a formula weight of about 6,000 daltons. In any embodiment, the block subunits of polyalkylene oxide in the TPU polymer can have a formula weight of about 8,000 daltons. In any embodiment, the block subunits of polyalkylene oxide in the TPU polymer can have a formula weight of about 12,000 daltons. In any embodiment, the block subunits of polyalkylene oxide in the TPU polymer can have a formula weight of about 6,000 daltons, a formula weight of about 8,000 daltons, a formula weight of about 12,000 daltons, a formula weight of about 6,000 daltons, or a mixture of block subunits having any two or more of the foregoing formula weights.

Composite nonwoven fabrics of the present disclosure comprise a population of staple fibers that are thermally bonded to the meltblown fibers. In any embodiment, the staple fibers may comprise staple fibers. Staple fibers are characterized by having a determinate length. In any embodiment, individual staple fibers may have a length of about 25 mm to about 100 mm, inclusive. The population of staple fibers in the composite nonwoven fabric has an average fiber length of about 38 mm to about 64 mm, inclusive.

The staple fibers further are characterized by having an average diameter. In any embodiment, the staple fibers of the present disclosure have an average diameter of about 5 μm to about 30 μm. For example, a composite nonwoven fabric comprising rayon fibers can have an average rayon fiber diameter from about 9 μm to about 30 μm. For example, a composite nonwoven fabric comprising nylon fibers can have an average nylon fiber diameter from about 13 μm to about 19 μm.

The staple fibers used in a composite nonwoven fabric of the present disclosure can be selected from a variety of suitable materials. Nonlimiting examples of suitable staple fibers include cellulose fibers, regenerated cellulose fibers, polyester fibers, polypeptide fibers, hemp fibers, flax fibers, nylon fibers, and a mixture of any two or more of the foregoing fibers.

The staple fibers comprise a portion (i.e., percentage) of the total weight of the composite nonwoven fabric. In any embodiment, the dry weight percent ratio of the meltblown fibers to the staple fibers is between about 60:40 and about 95:5, inclusive. In any embodiment, the dry weight percent ratio of the meltblown fibers to the staple fibers is between about 70:30 and about 95:5, inclusive. In any embodiment, the dry weight percent ratio of the meltblown fibers to the staple fibers is between about 80:20 and about 95:5, inclusive. In any embodiment, the dry weight percent ratio of the meltblown fibers to the staple fibers is between about 80:20 and about 90:10, inclusive. In any embodiment, the dry weight percent portion of the staple fibers in a composite nonwoven fabric of the present disclosure is about 15%, about 25%, about 30%, or about 40%

A composite nonwoven fabric of the present disclosure absorbs water and a variety of aqueous solutions having solutes dissolved therein. In any embodiment, the nonwoven fabrics are capable of absorbing bodily fluids (e.g., blood, serum, urine, and wound fluid), for example, which comprise salts, sugars, and/or proteins dissolved or suspended therein. In addition, the nonwoven fabrics are capable of absorbing other aqueous liquids such as, for example, lavage solutions (e.g., saline, normal saline, buffered saline, Ringer's solution) that are used to moisten and/or rinse wound sites. Lavage solutions typically contain solutes (e.g., sodium chloride, sodium lactate) dissolved therein.

In any embodiment, a composite nonwoven fabric of the present disclosure absorbs aqueous liquids (e.g., deionized water). The absorption of deionized water by the nonwoven fabric can be measured using a method that includes determining the mass of the dry fabric, immersing the fabric in deionized water, allowing the fabric to absorb the water until it is saturated, removing any excess water, and determining the mass of the water-saturated fabric. A full description of the absorption test is set forth in the Aqueous Solution Absorption Test disclosed herein. In any embodiment, the nonwoven fabric absorbs at least about 1990 grams of deionized water per gram of the fabric according to the Aqueous Solution Absorption Test disclosed herein. In any embodiment, the nonwoven fabric absorbs up to about 2175 grams of deionized water per gram of the fabric according to the Aqueous Solution Absorption Test disclosed herein.

In addition to absorbing water, in any embodiment, a composite nonwoven fabric of the present disclosure absorbs an aqueous solution comprising an ionic solute. A nonlimiting example of an aqueous solution comprising an ionic solute is normal saline (0.90% w/v NaCl in water). The absorption of normal saline by the nonwoven fabric can be measured using a method that includes determining the mass of the dry fabric, immersing the fabric in normal saline solution, allowing the fabric to absorb the solution until it is saturated, removing any excess solution, and determining the mass of the solution-saturated fabric. A full description of the absorption test is set forth in the Aqueous Solution Absorption Test disclosed herein. In any embodiment, the nonwoven fabric absorbs at least about 1925 grams of normal saline per gram of the fabric according to the Aqueous Solution Absorption Test disclosed herein. In any embodiment, the nonwoven fabric absorbs up to about 2080 grams of normal saline per gram of the fabric according to the Aqueous Solution Absorption Test disclosed herein.

Another example of an aqueous solution comprising an ionic solute is Ringer's solution. The absorption of normal saline by a nonwoven fabric of the present disclosure can be measured using a method that includes determining the mass of the dry fabric, immersing the fabric in Ringer's solution, allowing the fabric to absorb the solution until it is saturated, removing any excess solution, and determining the mass of the solution-saturated fabric. A full description of the absorption test is set forth in the Aqueous Solution Absorption Test disclosed herein. In any embodiment, the nonwoven fabric absorbs at least about 1880 grams of Ringer's solution per gram of the fabric according to the Aqueous Solution Absorption Test disclosed herein. In any embodiment, the nonwoven fabric absorbs up to about 2028 grams of normal saline per gram of the fabric according to the Aqueous Solution Absorption Test disclosed herein.

In any embodiment, one gram of the nonwoven fabric of the present disclosure absorbs at least about 80% as much normal saline as the amount of deionized water absorbs deionized water it typically absorbs. In any embodiment, one gram of the nonwoven fabric of the present disclosure absorbs at least about 90% as much normal saline as the amount of deionized water absorbs deionized water it typically absorbs. In a preferred embodiment, one gram of the nonwoven fabric of the present disclosure absorbs at least about 95% as much normal saline as the amount of deionized water absorbs deionized water it typically absorbs. In a more-preferred embodiment, one gram of the nonwoven fabric of the present disclosure absorbs at least about 97% as much normal saline as the amount of deionized water absorbs deionized water it typically absorbs.

In another aspect, the present disclosure provides an article comprising any embodiment of the composite nonwoven fabric disclosed herein. The article comprising the nonwoven fabric can be used for a variety of purposes including, for example, dressing a wound, treating a wound site, wiping a surface (e.g., an inanimate surface or a tissue surface such as skin, for example). Advantageously, the article comprising the composite nonwoven fabric can be used to absorb a variety of aqueous liquids that are present on a surface.

Ionic polymers (e.g., polyacrylates) are used in superabsorbent articles (e.g., diapers, wound dressings) in order to absorb bodily fluids. In addition to absorbing water, the ionic polymers tend to absorb other aqueous liquids (e.g., liquids that contain ionic moieties such as salts, for example), although generally not as well. Without being bound by theory, it is believed this is because the charges on those ionic polymers (e.g., negatively-charged carboxylate groups in acrylate polymers) repel and, thus, the negative charges of the ionic polymers are usually neutralized with positively-charged counter-ions such as sodium, for example. Upon contact with an aqueous liquid, the sodium ions are hydrated, thereby reducing their attraction to the carboxylate ions (e.g., due to the high dielectric constant of water). This reduced attraction permits the counter-ions to move freely within the polymer network, potentially increasing the osmotic pressure within the hydrated polymer gel. The mobile positive counter-ions cannot leave the gel, however, because they remain weakly attracted to the negatively-charged polymer backbone. Consequently, the ions trapped by the weak forces in the hydrated polymer gel create an osmotic potential within the polymer gel. This osmotic potential significantly favors the absorption of water and can significantly hinder the absorption of ionic solutions. Thus, the maximum swelling of these ionic polymer gels will occur in deionized water. Body fluids such as urine contain ions such as sodium, for example, and therefore are not absorbed as well as deionized water by these (ionic) superabsorbent polymers.

In contrast to conventional superabsorbent articles, in any embodiment, the inventive composite nonwoven articles of the present disclosure can be fabricated using a thermoplastic polyurethane that comprises a relatively high content of nonionic (e.g., alkylene oxide) units. Advantageously, this construction renders the nonwoven article able to absorb substantially similar volumes of pure aqueous solutions (e.g., deionized water) and aqueous solutions containing ionic solutes (e.g., NaCl) at concentrations similar to those found in bodily fluids.

Returning to the drawings, FIG. 2 shows one embodiment of an article 100 comprising a composite nonwoven fabric 152 according to the present disclosure. In any embodiment, an article according to the present disclosure may comprise a plurality of layers of nonwoven fabric. Each layer of the plurality of layers may comprise the same nonwoven fabric or at least one layer of the plurality of layers may comprise a different nonwoven fabric. In any embodiment, each layer of the plurality may comprise a composite nonwoven fabric according to the present disclosure. FIG. 3 shows one embodiment of an article 200 comprising a plurality of layers according to the present disclosure. The article 200 comprises a first layer 150 that comprises a first composite nonwoven fabric 152 and a second layer 160 that comprises a second composite nonwoven fabric 162, each composite nonwoven fabric being fabricated according to the present disclosure. The first layer 150 is coupled to the second layer 160 via any suitable means such as, for example, thermal bonding, adhesive bonding, stitching, stapling, needlepunching, calendaring, or a combination thereof.

In any embodiment, the composite nonwoven fabric 152 of each of the plurality of layers (e.g., first layer 150 and second layer 160) of an article (e.g., article 200) may be the substantially the same (e.g., compositionally (e.g., chemical composition, ratio of binding fibers to staple fibers) and/or physically (e.g., thickness, basis weight, area, average effective fiber diameter, average fiber length)) as the composite nonwoven fabric 162. In any embodiment, the composite nonwoven fabric 152 of each of the plurality of layers (e.g., first layer 150 and second layer 160) of an article (e.g., article 200) may be substantially different (e.g., compositionally (e.g., chemical composition, ratio of binding fibers to staple fibers) and/or physically (e.g., thickness, basis weight, area, average effective fiber diameter, average fiber length)) with respect to the composite nonwoven fabric 162.

An article according to the present disclosure has a basis weight. In any of the above embodiments, the article of the present disclosure may have a basis weight of about 20 g/m² to about 200 g/m², inclusive. In any embodiment, the article of the present disclosure may have a basis weight of about 50 g/m² to about 150 g/m², inclusive. In any embodiment, the article of the present disclosure may have a basis weight of about 800 g/m² to about 120 g/m², inclusive.

In any embodiment of an article according to the present disclosure, wherein the article comprises a plurality of layers of composite nonwoven fabric, the plurality of layers may have a basis weight of about 20 g/m² to about 200 g/m², inclusive. In any embodiment of an article according to the present disclosure, wherein the article comprises a plurality of layers of composite nonwoven fabric, the plurality of layers may have a basis weight of about 50 g/m² to about 150 g/m², inclusive. In any embodiment of an article according to the present disclosure, wherein the article comprises a plurality of layers of composite nonwoven fabric, the plurality of layers may have a basis weight of about 80 g/m² to about 120 g/m², inclusive. In any embodiment of an article according to the present disclosure, wherein the article comprises a plurality of layers of composite nonwoven fabric, the plurality of layers may have a basis weight of about 100 g/m².

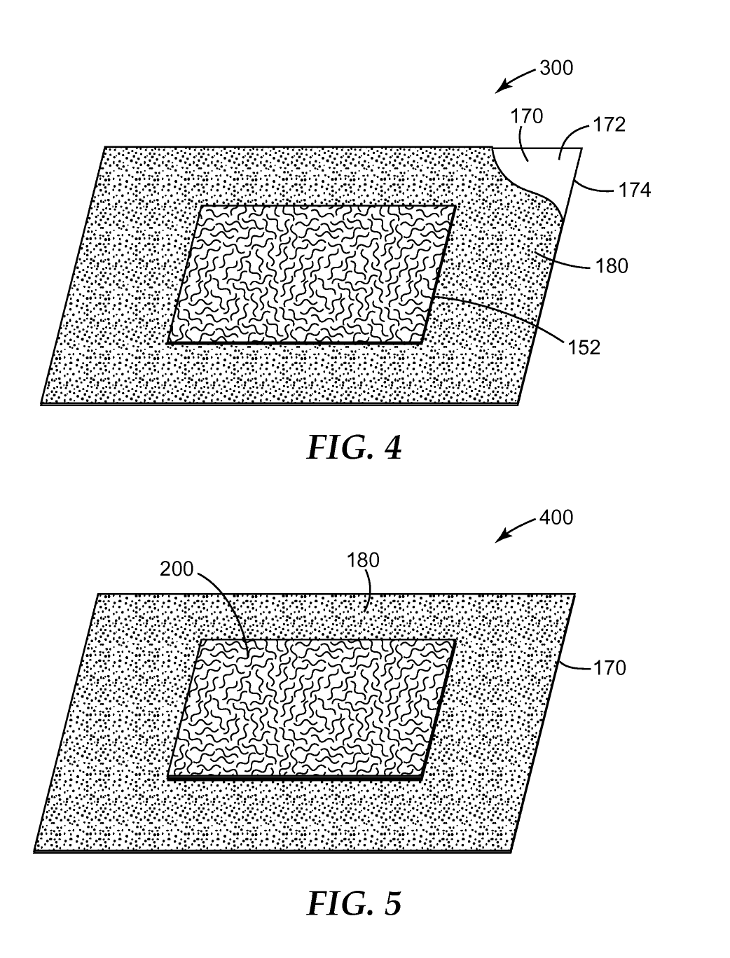

In any embodiment, an article according to the present disclosure comprises a backing layer. FIG. 4 shows an article 300 comprising a composite nonwoven fabric 152 and a backing layer 170 bonded thereto. In any embodiment, the composite nonwoven fabric 152 can be bonded to the backing layer 170 by any means known in the art such as, for example, thermal bonding, adhesive bonding, powdered binder, needlepunching, calendering, or a combination thereof. The composite nonwoven fabric 152 can be any embodiment of the composite nonwoven fabric disclosed herein.

FIG. 5 shows an article 400 comprising a backing layer 170 and the article 200 (of FIG. 3). The article 200 comprises a plurality of layers, both layers comprising a composite nonwoven fabric as described above. The nonwoven fabric article 200 is adhered to the backing layer 170 via an adhesive 180 coated on the backing layer. Examples of suitable adhesives 180 are described below.

The backing layer 170 can be fabricated from a variety of materials. Typically, the backing layer 170 is relatively thin (e.g., about 0.3 mm to about 3.0 mm thickness). In any embodiment, the backing layer may be fabricated from a material that substantially resists the passage of aqueous liquids therethrough.

Suitable backing materials for backing layer 170 include, for example, nonwoven fibrous webs, woven fibrous webs, knits, films and other familiar backing materials. The backing materials are typically translucent or transparent polymeric elastic films. The backing can be a high moisture vapor permeable film backing. U.S. Pat. No. 3,645,835; the disclosure of which is hereby incorporated by reference in its entirety; describes methods of making such films and methods for testing their permeability.

The backing advantageously should transmit moisture vapor at a rate equal to or greater than human skin. In some embodiments, the adhesive coated backing layer transmits moisture vapor at a rate of at least 300 g/m²/24 hrs/37° C./100-10% RH, frequently at least 700 g/m²/24 hrs/37° C./100-10% RH, and most typically at least 2000 g/m²/24 hrs/37° C./100-10% RH using the inverted cup method.

The backing layer 170 is generally conformable to anatomical surfaces. As such, when the backing layer 170 is applied to an anatomical surface, it conforms to the surface even when the surface is moved. The backing layer 170 is also conformable to animal anatomical joints. When the joint is flexed and then returned to its unflexed position, the backing layer 170 can be made such that it stretches to accommodate the flexion of the joint, but is resilient enough to continue to conform to the joint when the joint is returned to its unflexed condition.

A description of this characteristic of backing layers 170 for use with the present invention can be found in issued U.S. Pat. Nos. 5,088,483 and 5,160,315, the disclosures of which are hereby incorporated by reference in their entirety. Specific suitable backing materials are elastomeric polyurethane, co-polyester, or polyether block amide films. These films combine the desirable properties of resiliency, high moisture vapor permeability, and transparency found in backings.

Nonlimiting examples of suitable backing layer materials include a woven fabric, a knitted fabric, a foam (e.g., a CO₂-expanded polystyrene foam) layer, a film (e.g., a polyurethane film), a paper layer, an adhesive-backed layer, or a combination thereof. In any embodiment, the backing material can be sufficiently clear to permit visualization of objects through the backing layer.

Returning to FIG. 4, the backing layer 170 comprises a first major surface 172 and a second major surface 174 opposite the first major surface. In any embodiment, the backing layer 170 is bonded to the composite nonwoven fabric (e.g., layer 150 of composite nonwoven fabric) using a variety of bonding means known in the art. Nonlimiting examples of suitable means for bonding the backing layer 170 to the first layer 150 of the composite nonwoven fabric include thermal bonding, adhesive bonding, powdered binder, needlepunching, calendering, or any combination thereof.

In a preferred embodiment, the backing layer 170 is bonded to the composite nonwoven fabric (layer 150) via a pressure-sensitive adhesive. As illustrated in FIG. 4, at least a portion of the first major surface 172 has an optional adhesive layer 180 disposed (e.g., via coating processes that are well-known in the art) thereon. The adhesive layer 180 functions to bond the composite nonwoven fabric 152 to the backing layer 170.

Various pressure sensitive adhesives can be used to form adhesive layer 180 on the backing layer 170 to make the backing layer adhesive. The pressure sensitive adhesive is usually reasonably skin compatible and “hypoallergenic”, such as the acrylate copolymers described in U.S. Pat. No. RE 24,906, the disclosure of which is hereby incorporated by reference in its entirety. Particularly useful is a 97:3 iso-octyl acrylate:acrylamide copolymer, as is 70:15:15 isooctyl acrylate:ethyleneoxide acrylate:acrylic acid terpolymer described in U.S. Pat. No. 4,737,410; the disclosure of which is hereby incorporated by reference in its entirety; is suitable. Additional useful adhesives are described in U.S. Pat. Nos. 3,389,827; 4,112,213; 4,310,509; and 4,323,557; the disclosures of which are hereby incorporated by reference in their entirety. Inclusion of medicaments or antimicrobial agents in the adhesive is also contemplated, as described in U.S. Pat. Nos. 4,310,509 and 4,323,557, both of which are also hereby incorporated by reference in their entirety.

In the illustrated embodiment, the composite nonwoven fabric defines a first area and the backing layer defines a second area that is larger than the first area. The second area is shaped and dimensioned such that at least a portion (e.g., peripheral portion) of the second area extends outside the first area. Thus, the peripheral portion can be adhered to a surface (e.g., a skin surface) via the adhesive layer, thereby securing (e.g., reversibly securing) the article to the surface (e.g., the skin surface, not shown).

### EXEMPLARY EMBODIMENTS

Embodiment A is a composite nonwoven fabric, comprising:

a population of meltblown fibers comprising an aliphatic polyether thermoplastic polyurethane polymer having at least about 80% (w/w) polyalkylene oxide; and

a population of staple fibers intermixed and entangled therewith.

Embodiment B is the composite nonwoven fabric of Embodiment A, wherein the aliphatic polyether thermoplastic polyurethane polymer has at least about 90% (w/w) polyalkylene oxide.

Embodiment C is the composite nonwoven fabric of Embodiment A or Embodiment B, wherein the polyalkylene oxide comprises poly(ethylene oxide).

Embodiment D is the composite nonwoven fabric of any one of the preceding embodiments, wherein the staple fibers are selected from the group consisting of cellulose fibers, regenerated cellulose fibers, polyester fibers, polypeptide fibers, hemp fibers, flax fibers, nylon fibers, and a mixture of any two or more of the foregoing fibers.

Embodiment E is the composite nonwoven fabric of any one of the preceding Embodiments, wherein the staple fibers comprise staple fibers.

Embodiment F is the composite nonwoven fabric of any one of the preceding Embodiments, wherein the average length of the staple fibers is about 25 mm to about 100 mm, inclusive.

Embodiment G is the composite nonwoven fabric of any one of the preceding Embodiments, wherein a weight percent ratio of the meltblown fibers to the staple fibers is about 60:40 to about 95:5.

Embodiment H is the composite nonwoven fabric of any one of the preceding Embodiments, wherein the average diameter of the meltblown fibers is about 2 μm to about 25 μm.

Embodiment I is the composite nonwoven fabric of any one of the preceding Embodiments, wherein the polyalkylene oxide comprises polyethylene oxide or polypropylene oxide.

Embodiment J is the composite nonwoven fabric of any one of the preceding Embodiments, wherein the polyurethane polymer comprises block subunits of polyethylene oxide, wherein the block subunits have an average formula weight of about 6,000 daltons to about 20,000 daltons.

Embodiment K is the composite nonwoven fabric of any one of the preceding Embodiments wherein, according to the Nonwoven Absorbency Test defined herein, the fabric absorbs at least about 1880 grams of Ringers Solution per gram of the fabric.

Embodiment L is the composite nonwoven fabric of any one of the preceding Embodiments wherein, according to the Nonwoven Absorbency Test defined herein, the fabric absorbs at least about 1990 grams of deionized water per gram of the fabric.

Embodiment M is the composite nonwoven fabric of any one of the preceding Embodiments wherein, according to the Nonwoven Absorbency Test defined herein, the fabric absorbs at least about 1925 grams of normal saline solution per gram of the fabric.

Embodiment N is the composite nonwoven fabric of any one of Embodiments A through M;

wherein a first amount of deionized water is absorbed per gram of dry composite nonwoven fabric and a second amount of Ringer's Solution is absorbed per gram of dry composite nonwoven fabric, both determined by the Nonwoven Absorbency Test defined herein;

wherein the second amount is at least about 80% of the first amount.

Embodiment O is the composite nonwoven fabric of any one of Embodiments A through N, wherein the second amount is at least about 90% of the first amount.

Embodiment P is the composite nonwoven fabric of any one of Embodiments A through O;

wherein a first amount of deionized water is absorbed per gram of dry composite nonwoven fabric and a third amount of normal saline solution is absorbed per gram of dry composite nonwoven fabric, both determined by the Nonwoven Absorbency Test defined herein;

wherein the third amount is at least about 80% of the first amount.

Embodiment Q is the composite nonwoven fabric of Embodiment P, wherein the third amount is at least about 90% of the first amount.

Embodiment R is an article comprising the composite nonwoven fabric of any one of Embodiment A through Q.

Embodiment S is the article of Embodiment R, wherein the article comprises a plurality of layers, wherein at least one of the plurality of layers comprises the composite nonwoven fabric.

Embodiment T is the article of Embodiment S, wherein a first layer of the plurality of layers is coupled to a second layer of the plurality of layers.

Embodiment U is the article of Embodiment T, wherein the first layer is coupled to the second layer via thermal bonding, adhesive bonding, stitching, stapling, needlepunching, calendaring, or a combination thereof.

Embodiment V is the article of any one of Embodiments R through U, wherein the article has a basis weight of about 20 g/m² to about 200 g/m².

Embodiment W is the article of any one of Embodiments R through V, further comprising a backing layer having a first major surface and a second major surface opposite the first major surface, wherein the composite nonwoven fabric is bonded to the first major surface.

Embodiment X is the article of Embodiment W, wherein the backing layer comprises a nonwoven fabric, a woven fabric, a knitted fabric, a foam layer, a film, a paper layer, or a combination thereof.

Embodiment Y is the article of Embodiment W or Embodiment X, wherein the backing layer is bonded to the composite nonwoven fabric using thermal bonding, adhesive bonding, powdered binder, needlepunching, calendering, or a combination thereof.

Embodiment Z is the article of any one of Embodiments W through Y, wherein the composite nonwoven fabric defines a first area and the backing layer defines a second area that is shaped and dimensioned such that at least a portion of the second area extends outside the first area.

Embodiment AA is the article of Embodiment Z, wherein the first major surface of the portion comprises an adhesive layer coated thereon.

Embodiment BB is the article of any one of the preceding claims, wherein at least a portion of the population of staple fibers is thermally bonded to the meltblown fibers.

Objects and advantages of this invention are further illustrated by the following examples, but the particular materials and amounts thereof recited in these examples, as well as other conditions and details, should not be construed to unduly limit this invention.

### EXAMPLES

Materials

Materials used for the examples are shown in Table 1.

TABLE 1
Materials List

| Material | Description | Source |
|---|---|---|
| Polyurethane (PU) | TG-2000 Polyurethane | Lubrizol (Wickliffe, OH) |
| TENCEL fibers | 1.7 denier (1.9 decitex) | Lenzing AG, Lenzing, |
| | TENCEL fibers | Austria |
| Melty fiber | 2 denier (2.2 decitex) | Huvis; Seoul, South |
| | bicomponent polyester | Korea |
| | LMF fiber | |

Test Methods

Test Solutions

Ringers solution was prepared by mixing 16.58 g NaCl and 0.72 g CaCl₂ in 2000 g distilled water.

Aqueous Solution Adsorption Test

A dry sample (5.1 cm×5.1 cm) of the nonwoven fabric to be tested was cut, weighed, and placed in a Petri dish. Test solution (40 g) was added to the Petri dish to cover the nonwoven fabric sample. The nonwoven fabric sample was allowed to passively absorb the test solution at 37° C. for 30 minutes. The test solution was then decanted from the Petri dish. The nonwoven fabric sample was then removed from the Petri dish with fingers and, while holding a corner, the final drop of water was removed with an absorbent tissue. The liquid-saturated fabric was then re-weighed and the % absorption ((grams test solution absorbed/grams of dry non-woven)×100) was recorded. The mean and standard deviations of the masses for each of 8 replicate non-woven fabric samples were recorded.

Production of Staple Fiber Web

A random card machine was used to lay down a web of 95% (by weight) TENCEL fibers and 5% Melty fiber. The web was then thermally point bonded to provide integrity.

### Comparative Example 1

A nonwoven fabric was made from PU using the equipment shown in FIG. 1 and the process essentially as described in U.S. Pat. No. 4,118,531. The PU polymer was meltblown at a temperature of 200° C. and at a rate of 1.0 pounds per hour per inch of die width. The feed rate of the TENCEL rayon fibers that contained 5% by weight of 2 denier Huvis melty fiber was adjusted to provide the samples listed in Table 2. The staple fibers consisted of TENCEL rayon fibers having an average fiber length of about 38 mm and an average fiber diameter of about 12.6 μm.

### Examples 1-5

Nonwoven fabrics were made using the equipment and conditions described in Comparative Example 1. The input flow rates of the polyurethane polymer and staple fibers were adjusted to yield the web compositions and basis weights shown in Table 2.

TABLE 2
Non-woven web compositions.

| | |
|---|---|
| Fiber Component | |
| (weight percent) | Web Basis |

| Sample | PU | Staple fibers | Weight (g/m²) |
|---|---|---|---|
| Comparative | 100 | 0 | 110 |
| Example 1 | | | |
| Example 1 | 85 | 15 | 130 |
| Example 2 | 70 | 30 | 65 |
| Example 3 | 69 | 31 | 97 |
| Example 4 | 71 | 29 | 35 |
| Example 5 | 60 | 40 | 25 |

Absorption of aqueous liquids by non-woven fabrics of Comparative Example 1 and Examples 1-5.

The non-woven fabrics of Comparative Example 1 and Examples 1-5 were cut into 5.1 cm×5.1 cm pieces and the pieces were subjected to the Aqueous Solution Absorption Test described above. Each fabric was tested for its ability to absorb distilled water and Ringers Solution. The results are shown in Table 3.

TABLE 3
Aqueous Solution Absorption Test results. The data are
reported as the mean ± standard deviation of eight
replicate samples of each fabric.

| | Absorptivity | | |
|---|---|---|---|
| | (grams solution per gram dry web) | | |
| Sample | Distilled Water | Ringers Solution | Normal Saline |
| Comparative | | | |
| Example 1 | | | |
| Example 1 | 2037 ± 48 | 1985 ± 85 | 2070 ± 62 |
| Example 2 | 2077 ± 98 | 2000 ± 81 | 2052 ± 96 |
| Example 3 | 2175 ± 57 | 1972 ± 90 | 2080 ± 63 |
| Example 4 | 1993 ± 51 | 1882 ± 126 | 1925 ± 80 |
| Example 5 | 2108 ± 86 | 2028 ± 110 | 2020 ± 74 |

The data indicate that all of the nonwovens absorbed at least about 95% as much saline per gram dry web as compared to the amount of distilled water they absorbed. In addition, the data indicate that all of the nonwovens absorbed at least about 90% as much saline per gram dry web as compared to the amount of distilled water they absorbed.

The complete disclosure of all patents, patent applications, and publications, and electronically available material cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

Various modifications may be made without departing from the spirit and scope of the invention. These and other embodiments are within the scope of the following claims.

1. A composite nonwoven fabric, comprising:
a population of meltblown fibers comprising an aliphatic polyether thermoplastic polyurethane polymer having at least about 80% (w/w) polyalkylene oxide; and
a population of staple fibers intermixed and entangled therewith.

2. The composite nonwoven fabric of claim 1, wherein the staple fibers are selected from the group consisting of cellulose fibers, regenerated cellulose fibers, polyester fibers, polypeptide fibers, hemp fibers, flax fibers, nylon fibers, and a mixture of any two or more of the foregoing fibers.

3. (canceled)

4. The composite nonwoven fabric of claim 1, wherein the average length of the staple fibers is about 5 mm to about 30 mm.

5. The composite nonwoven fabric of claim 1, wherein a weight percent ratio of the meltblown fibers to the staple fibers is about 60:40 to about 95:5.

6. The composite nonwoven fabric of claim 1, wherein the average diameter of the meltblown fibers is about 2 μm to about 25 μm.

7. The composite nonwoven fabric of claim 1, wherein the polyalkylene oxide comprises polyethylene oxide or polypropylene oxide.

8. The composite nonwoven fabric of claim 1, wherein the polyurethane polymer comprises block subunits of polyethylene oxide , wherein the block subunits have an average formula weight of about 6,000 daltons to about 20,000 daltons.

9. The composite nonwoven fabric of claim 1, according to the Nonwoven Absorbency Test defined herein, the fabric absorbs at least about 1880 grams of Ringer's solution per gram of the fabric.

10. The composite nonwoven fabric of claim 1, according to the Nonwoven Absorbency Test defined herein, the fabric absorbs at least about 1925 grams of normal saline solution per gram of the fabric.

11. The composite nonwoven fabric of claim 9 wherein, according to the Nonwoven Absorbency Test defined herein, the fabric absorbs at least about 1990 grams of deionized water per gram of the fabric.

12. An article comprising the composite nonwoven fabric of claim 1.

13. The article of claim 12, wherein the article comprises a plurality of layers, wherein at least one of the plurality of layers comprises the composite nonwoven fabric.

14. The article of claim 13, wherein a first layer of the plurality of layers is coupled to a second layer of the plurality of layers.

15. The article of claim 14, wherein the first layer is coupled to the second layer via thermal bonding, adhesive bonding, stitching, stapling, needlepunching, calendaring, or a combination thereof.

16. The article of claim 12, wherein the article has a basis weight of about 20 g/m² to about 200 g/m².

17. The article of claim 12, further comprising a backing layer having a first major surface and a second major surface opposite the first major surface, wherein the composite nonwoven fabric is bonded to the first major surface.

18. The article of claim 17, wherein the backing layer comprises a nonwoven fabric, a woven fabric, a knitted fabric, a foam layer, a film, a paper layer, or a combination thereof.

19. The article of claim 17, wherein the backing layer is bonded to the composite nonwoven fabric using thermal bonding, adhesive bonding, powdered binder, hydroentangling, needlepunching, calendering, or a combination thereof.

20. The article of claim 17, wherein the composite nonwoven fabric defines a first area and the backing layer defines a second area that is shaped and dimensioned such that at least a portion of the second area extends outside the first area.
